(19) United States
(12) Patent Application Publication
(10) Pub. No.: US 2004/0243253 A1
(43) Pub. Date: Dec. 2, 2004

(54) Prosthesis or orthesis for a member of a human body

(75) Inventors: Jan Constant Cool, Pijnacker (NL); Frederik Lambert Silvio Te Riele, Hengelo (NL); Edsko Evert Geert Hekman, Enschede (NL)

Correspondence Address: PEACOCK MYERS AND ADAMS P C, P O BOX 26927, ALBUQUERQUE, NM, 871256927

(73) Assignee: Stichting voor de Technische Wetenschappen, Utrecht (NL)

(21) Appl. No.: 10/762,628

(22) Filed: Jan. 22, 2004

Related U.S. Application Data
(63) Continuation-in-part of application No. PCT/NL02/00497, filed on Jul. 23, 2002.

(30) Foreign Application Priority Data
Jul. 23, 2001 (NL) 1018616

(51) Int. Cl. A61F 2/66; A61F 5/052

(52) U.S. Cl. 623/52; 602/16; 623/55

(57) ABSTRACT

A prosthesis or orthesis for a member of a human body, comprising limb-forming components which components are connected by pivoting couplings and at least one first element whose ends are coupled with components that are distinguishable from each other, wherein the first spring element spans at least two pivoting couplings.

The invention relates to a prosthesis or orthesis for a member of a human body, comprising limb-forming components, which components are connected by pivoting couplings, and at least one first spring element whose ends are coupled with components that are distinguishable from each another.

Such a prosthesis is known from the Dutch patent application NL-A-1010209. In the known prosthesis a spring means is used coupling a toe portion with a heel portion. A damper is provided to complete the thus formed mass spring system.

DE-C-309066 relates to a prosthetic foot comprised of various parts that are connected with each other and in which, in order to restrict the movability of the middle part of the foot, the same is pivotingly connected with two levers connected on the one hand with the lower leg and on the other hand with the middle part of the foot. The object of the construction is to provide a restriction to the movability of the parts of the foot in relation to one another.

WO 00/23017 relates to a prosthesis fitted to the lower leg and comprising an energy storage element in the form of a leaf spring, with a lever being pivotingly coupled to the lower side of said leaf spring. Between the lever and the leaf spring an energy transmission element is provided in the form of an inelastic cord. In this way the movement of the lever translates into a corresponding energy storage in the leaf spring.

The known prosthesis and also other prior art prostheses and ortheses have disadvantages which the present invention is intended to eliminate.

One problem of the known prostheses and ortheses is that they do not allow the user to assure a natural gait. On the one hand this is to do with the suboptimal energy efficiency of known prostheses ortheses, and on the other hand this is do with the inadequate support that the known prostheses and ortheses afford the body of the user during walking. This is particularly relevant during the push-off. The poor functioning of the known prosthesis 5 results in the user being moderately comfortable when walking and becoming very fatigued.

In a first aspect of the invention, the prosthesis and orthesis referred to in the preamble is according to the inventors characterized in that a coupling element is provided spanning at least two pivoting couplings of a limb-forming component or part. In this way an energetic, substantially neutral use of the prosthesis or orthesis can be achieved for which no or very little external energy needs to be supplied. The energy that during the walking motion is stored in the first spring element can be made available with a high conversion efficiency to a further spring element that may form a part of the prosthesis or orthesis.

The idea of the invention outlined above can be used for the lower leg where a knee-ankle coupling has to be provided but, for example, also for a prosthesis or orthesis for the upper leg where the coupling between the torso and the knee has to be provided. Other parts of the body may also be substituted or supported by a prosthesis or orthesis in the manner proposed by the invention; in this connection the application as prosthetic foot may be mentioned. The invention will be further elucidated with reference to such a prosthetic foot. However, the invention is obviously not limited thereto.

In a further aspect of the invention, the prosthetic foot or orthesis is characterized in that the parts comprise a lower leg portion, a heel portion, a front portion of the foot and a toe portion, wherein the front portion of the foot at either side ends in an ankle hinge and a toe hinge, respectively, for the coupling of on the one hand the heel portion and the lower leg portion, and on the other end the toe portion, and in that the coupling element spans the pivot couplings of the front portion of the foot.

The coupling element preferably spans the pivoting coupling of the front portion of the foot in such a manner that the same is connected on the one hand with the toe portion and on the other hand with a selection made from the group comprised of the lower leg portion and the heel portion.

Preferably the first coupling element is connected on the one hand with the toe portion and on the other hand with the lower leg portion. This allows the prosthetic foot or orthesis to be embodied relatively simply making it even possible to integrate the front portion of the foot and the heel part.

The prosthesis or orthesis may be constructed relatively simply by allowing the coupling element and the first spring element to coincide, that is to say by embodying the coupling element as spring element.

In still another aspect of the invention the prosthesis or orthesis is characterized in that the heel portion and the lower leg portion share the ankle hinge of the front portion of the foot, while also being mutually coupled via a second spring element. This second spring element can then fulfil the above-mentioned energy-exchange function with the first spring element.

Herein below the invention will be further elucidated with reference to a non-limiting exemplary embodiment of a prosthetic foot and with reference to the accompanying drawing.

The drawing shows in:

FIG. 1 a schematic illustration of a prosthetic foot according to the invention;

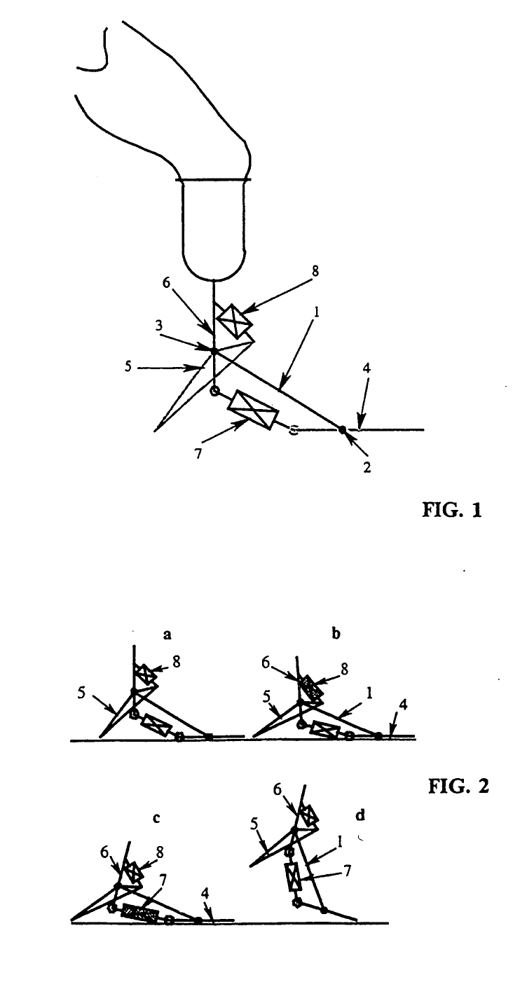

FIG. 2 a schematic illustration of various stages of use a to d of the prosthetic foot according to the invention; and

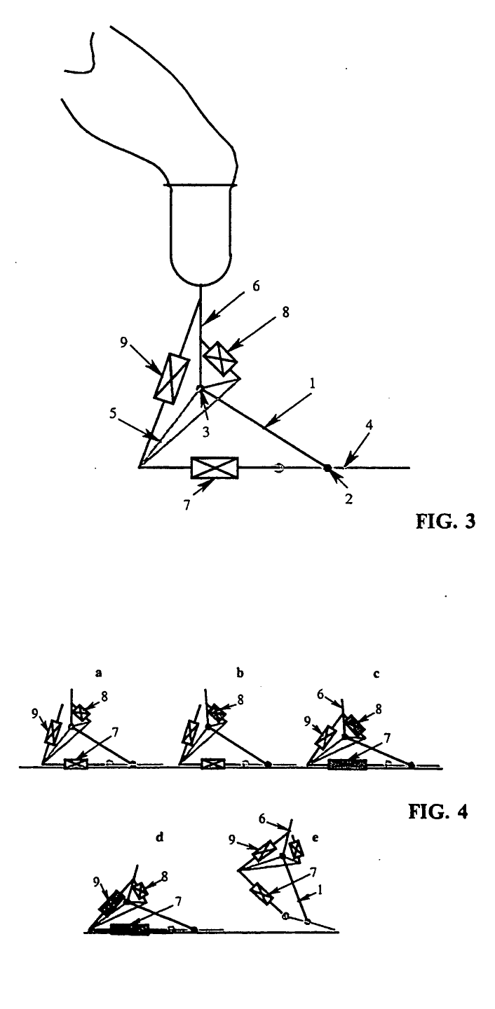

FIG. 3 a schematic illustration of an alternative embodiment of the prosthetic foot according to the invention; and

FIG. 4 various stages of use of the prosthetic foot according to FIG. 3.

Identical reference numbers used in the figures refer to identical parts.

Referring first to FIG. 1, a front part of a foot is shown indicated by reference number 1, ending in a toe hinge 2 and an ankle hinge 3. By means of toe hinge 2 a toe portion 4 is coupled with the front portion of the foot 1. Ankle hinge 3 couples the front portion of the foot 1 and the heel portion 5. Ankle hinge 3 also couples a lower leg portion 6. In this embodiment shown in FIG. 1, the lower leg portion 6 extends past the ankle hinge 3 and via a so-called plantar spring element 7, this lower leg portion 6 is coupled with the toe portion 4. In this way the plantar spring element 7 spans both the ankle hinge 3 and the toe hinge 2. The heel portion 5 is further coupled with the lower leg portion 6 by a so-called dorsal spring element 8.

The use of the embodiment of the prosthetic foot according to the invention discussed with reference to FIG. 1 is schematically shown in FIGS. 2a to d. In situation a the prosthetic foot is unloaded. In situation b, the heel portion 5 makes loaded contact with the ground, and the dorsal spring element 8 is under tension. The energy generated by the prosthetic foot doming down is thus stored in the dorsal spring element 8. This energy is at a maximum when the heel portion 5 has reached maximal plantar flexure (turned clockwise) in relation to the lower leg portion 6. In situation b, the lower leg portion 6 is shown to be tilted in relation to the vertical. However, this does not mean that the front portion of the foot 1 is necessarily always in a condition of plantar flexure in relation to the lower leg portion 6. When continuing the walking motion as shown in situation c, the user of the prosthetic foot will flex dorsally, which results in the release of the dorsal spring element 8 and the tensioning of the plantar spring element 7. The energy stored in the dorsal spring element 8 during placement of the prosthetic foot as shown in Sub-FIG. b is released, making it available for tensioning the plantar spring element 7, which provides the coupling between the toe portion 4 and the lower leg portion 6.

Sub-FIG. d shows the situation in which the amount of built-up plantar flexing moment developed by the plantar spring element 7 around the ankle hinge is such, that the heel portion 5 comes off the ground, while the front portion of the foot 1 undergoes plantar flexure in relation to the lower leg portion 6. This provides the desired elongation of the lower leg portion 6, with the resuit that the user's torso continues to be supported at a correct height. During the further push-off of the prosthetic foot the loads will decrease further so that the plantar spring element will be released also.

FIGS. 1 and 2 show the construction and working of the prosthetic foot, with the plantar spring element 7 providing the coupling between the toe portion 4 and the lower leg portion 6.

FIGS. 3 and 4 show the construction and working with the plantar spring element 7 being coupled between the toe portion 4 and the heel portion 5. In the construction shown in FIG. 3, the heel portion 5 then needs to be coupled to the lower leg portion 6 by means of a heel spring element 9. For the remainder, the mutual coupling between the lower leg portion 6, the front of the foot part 1, the heel portion 5 and the toe portion 4 is provided in a similar manner as that shown in the embodiment of FIG. 1. It should be noted at this point that the said coupling of the heel portion 5 with the lower leg portion 6 by means of the heel spring element 9 is realised using a switching coupling 10.

Situation a in FIG. 4 shows the prosthetic foot in the unloaded condition; the heel spring element 9 is uncoupled. Situation b shows that the heel portion 5 is in contact with the ground;. the dorsal spring 8 is under tension and, as shown in situation c, the plantar spring element 7 in turn also comes under tension, effecting the coupling of the heel spring element 9 with the lower leg portion 6. When subsequently dorsal flexure occurs as shown in situation d, the dorsal spring 8 is released, the heel spring element 9 comes under tension and the plantar spring 7 remains under tension. In the further rolling-off motion of the prosthetic foot, in which the front portion of the foot 1 undergoes plantar flexure in relation to the S lower leg portion 6, the plantar spring 7 and the heel spring element 9 are released. Eventually the heel spring element 9 will uncouple from the lower leg portion to allow the prosthetic foot to return to the unloaded condition (see situation a).

It should be noted that although the design of the prosthetic foot elucidated in FIGS. 3 and 4 illustrating the second embodiment requires a switching mechanism, this switching mechanism may also be omitted by suitably dimensioning the prosthetic foot. is The prosthesis and/or orthesis proposed in accordance with the invention shows that plantar flexure (straightening) of the prosthesis according to the invention can be realised, which makes it possible to achieve the necessary elongation of the leg in the push-off stage of the rolling-off motion of the foot. The combined application of the plantar spring element and the dorsal spring element as proposed in accordance with the invention allows the prosthetic foot according to the invention to be used at a very high energy conversion efficiency.

What is claimed is:

1. A prosthesis or orthesis for a member of a human body, comprising limb-forming components, which components are connected by pivoting couplings, and at least one first spring element whose ends are coupled with components that are distinguishable from each other, wherein a coupling element is provided spanning at least two of said pivoting couplings.

2. A prosthesis or orthesis according to claim 1, wherein the member is a foot, and further comprising a lower leg portion, a heel portion, a front portion of the foot and a toe portion, wherein the front portion of the foot at either side ends in an ankle hinge and a toe hinge, respectively, for the coupling of on the one hand the heel portion and the lower leg portion, and on the other end the toe portion, and wherein the coupling element spans the pivoting couplings of the front portion of the foot.

3. A prosthesis or orthesis according to claim 2, wherein the coupling element is connected on the one hand with the toe portion and on the other hand with a selection made from the group consisting of the lower leg portion and the heel portion.

4. A prosthesis or orthesis according to claim 2, wherein the coupling element is connected on the one hand with the toe portion and on the other hand with the lower leg portion.

5. A prosthesis or orthesis according to claim 4, wherein the front portion of the foot and the heel part are integrated to form a whole.

6. A prosthesis or orthesis according to claim 1, wherein the coupling element is the first spring element.

7. A prosthesis or orthesis according to claim 2 wherein the heel portion and the lower leg portion share the ankle hinge of the front portion of the foot, while also being mutually coupled via a second spring element.
